(19) United States
(12) Patent Application Publication
(10) Pub. No.: US 2019/0062689 A1
(43) Pub. Date: Feb. 28, 2019

(54) CULTURE MONITORING SYSTEM, CONTROL METHOD, AND COMPUTER-READABLE MEDIUM

(71) Applicant: OLYMPUS CORPORATION, Tokyo (JP)

(72) Inventors: Naohiro ARIGA, Tokyo (JP); Toshiyuki HATTORI, Tokyo (JP); Mina KOBAYASHI, Tokyo (JP); Ayumu SAKURAI, Tokyo (JP)

(73) Assignee: OLYMPUS CORPORATION, Tokyo (JP)

(21) Appl. No.: 16/100,175

(22) Filed: Aug. 9, 2018

(30) Foreign Application Priority Data
Aug. 28, 2017 (JP) 2017-163491

(51) Int. Cl. C12M 1/00; C12M 1/36; C12M 1/34; G06T 7/11; H04N 5/30

(52) U.S. Cl. CPC C12M 41/14; C12M 41/48; H04N 5/30; G06T 7/11; C12M 41/46

(57) ABSTRACT

A culture monitoring system includes: an image acquisition device that is arranged in an incubator and acquires an image of a biological sample cultured in the incubator; and a controller that controls the image acquisition device. The controller includes an estimator that estimates a state of a temperature of the biological sample with respect to a set temperature of the incubator, and a device controller that restricts an operation of the image acquisition device at least according to the state.

### CROSS REFERENCE TO RELATED APPLICATIONS

This application is based upon and claims the benefit of priority from prior Japanese Patent Application No. 2017-163491, filed Aug. 28, 2017, the entire contents of which are incorporated herein by this reference.

### BACKGROUND OF THE INVENTION

### Field of the Invention

Embodiments disclosed herein relate to a culture monitoring system, a control method, and a computer-readable medium.

### Description of the Related Art

Conventionally, a biological sample such as a cell is cultured in an incubator in order to maintain a specified culture environment. The growth state of a biological sample in culture is monitored regularly, but if the biological sample in culture is taken out of the incubator, the temperature of the biological sample will be decreased, which may result in affecting the growth of the biological sample negatively. In order to overcome this problem, a remote monitoring system has been proposed that monitors a biological sample in culture without taking it out of an incubator (see, for example, Japanese Patent No. 6063967).

### SUMMARY OF THE INVENTION

A culture monitoring system according to an aspect of the present invention includes: an image acquisition device that is arranged in an incubator, includes an image sensor that generates an image signal, and acquires, using the image sensor, an image of a biological sample cultured in the incubator; and a controller that includes a processor and controls the image acquisition device. The processor estimates a state of a temperature of the biological sample with respect to a set temperature of the incubator, and restricts an operation of the image acquisition device at least according to the estimated state.

A control method according to an aspect of the present invention is a method for controlling an image acquisition device that is arranged in an incubator, the control method including: estimating a state of a temperature of the biological sample with respect to a set temperature of the incubator; and restricting an operation of the image acquisition device at least according to the state, the image acquisition device acquiring an image of the biological sample cultured in the incubator.

A non-transitory computer-readable medium according to an aspect of the present invention has stored therein a program that causes a controller to execute a process, the controller controlling an image acquisition device that is arranged in an incubator and acquires an image of a biological sample cultured in the incubator, the process including: estimating a state of a temperature of the biological sample with respect to a set temperature of the incubator; and restricting an operation of the image acquisition device at least according to the state.

### BRIEF DESCRIPTION OF THE DRAWINGS

The present invention will be more apparent from the following detailed description when the accompanying drawings are referenced.

FIG. 1 illustrates a configuration of a culture monitoring system 1;

FIG. 2 illustrates a configuration of the inside of a housing of an image acquisition device 20;

FIG. 3 is a diagram for explaining an image acquisition method performed by the image acquisition device 20;

FIG. 4 is a diagram for explaining a focus operation performed by the image acquisition device 20 at the time of acquiring an image;

FIG. 5 is a block diagram that illustrates a hardware configuration of a controller 40;

FIG. 6 is a block diagram that illustrates a functional configuration of the controller 40;

FIG. 7 is a flowchart that illustrates an example of monitoring processing according to a first embodiment;

FIG. 8 is a sequence diagram that illustrates a communication of data between the controller 40 and the image acquisition device 20;

FIG. 9 illustrates an image acquisition point according to the first embodiment;

FIG. 10 illustrates an example of a change in a state of the image acquisition device 20 according to the first embodiment;

FIG. 11 illustrates a screen displayed in a stopping state;

FIG. 12 illustrates another example of the change in a state of the image acquisition device 20 according to the first embodiment;

FIG. 13 is a flowchart that illustrates an example of monitoring processing according to a second embodiment;

FIG. 14 illustrates an image acquisition point according to the second embodiment;

FIG. 15 is a flowchart that illustrates preparation processing according to a third embodiment;

FIG. 16 illustrates an example of a change in a state of the image acquisition device 20 according to the third embodiment;

FIG. 17 illustrates an example of a weighting coefficient list;

FIG. 18 is a flowchart that illustrates an example of monitoring processing according to a fourth embodiment;

FIG. 19 illustrates an image acquisition point according to the fourth embodiment;

FIG. 20 is a flowchart that illustrates an example of monitoring processing according to a fifth embodiment; and

FIG. 21 illustrates an image acquisition point according to the fifth embodiment.

### DESCRIPTION OF THE EMBODIMENTS

When an image acquisition device is provided within an incubator as disclosed in Japanese Patent No. 6063967, heat generated in the image acquisition device is transferred to a biological sample via a culture vessel, which may result in increasing the temperature of the biological sample above a set temperature of the incubator.

When the temperature of a biological sample is increased, the biological sample is cultured in a culture environment at a temperature that is different from the temperature of an intended culture environment. Further, when the temperature is increased excessively, the biological sample is damaged by heat.

Taking this into consideration, embodiments of the disclosure will now be described with reference to the drawings.

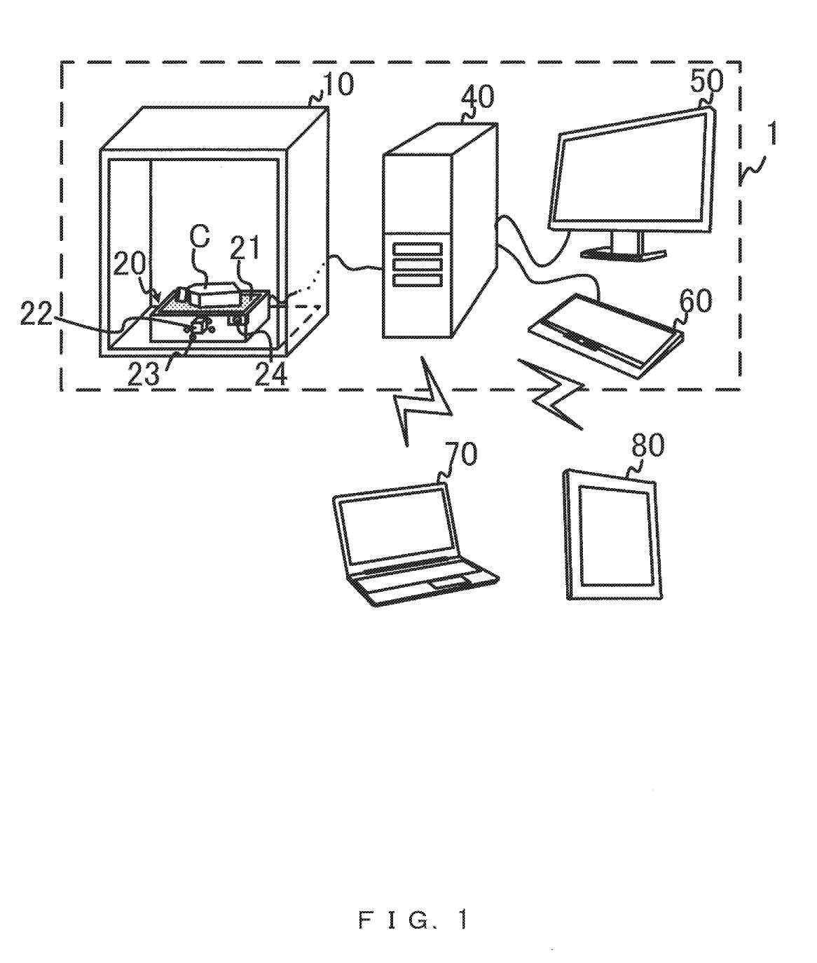

First, a culture monitoring system 1 is described with reference to FIG. 1. FIG. 1 illustrates a configuration of the culture monitoring system 1.

The culture monitoring system 1 is a system that monitors a biological sample cultured in an incubator 10. The biological sample is, for example, a cultured cell such as an adherent cell, and is accommodated in a flask C. The incubator 10 is a device that maintains or controls a culture environment (such as temperature and humidity) in which a biological sample is cultured. The culture vessel used to accommodate a biological sample is not limited to the flask C, and it may be, for example, a multi-well plate or a dish.

The culture monitoring system 1 includes an image acquisition device 20 that acquires an image of a biological sample cultured in the incubator 10, and a controller 40 that controls the image acquisition device 20. The image acquisition device 20 is arranged in the incubator 10.

The image acquisition device 20 and the controller 40 are connected to each other through a wireline cable such as a USB (universal serial bus) cable. However, it is sufficient if the image acquisition device 20 and the controller 40 can mutually communicate data, so they may be communicatively connected to each other wirelessly instead of being connected by wire.

The culture monitoring system 1 may further include a liquid crystal display 50 that is an example of a display device, and a keyboard 60 that is an example of an input device. Instead of the liquid crystal display 50, the culture monitoring system 1 may include, for example, an organic EL (OLED) display or a CRT display as a display device. Instead of or in addition to the keyboard 60, the culture monitoring system 1 may include, for example, a mouse, a joystick, or a touch panel as an input device.

A user of the culture monitoring system 1 may access the controller 40 of the culture monitoring system 1 through at least one of a wireless communication and a wired communication, using a client terminal such as a laptop computer 70, a tablet computer 80, and a smartphone. In this case, the client terminal operates as a display device and an input device on behalf of the liquid crystal display 50 and the keyboard 60.

The image acquisition device 20 includes a housing that has a top plate 21 that is transparent and flat. The flask C is arranged on the top plate 21. The top plate 21 is, for example, a glass plate. However, the top plate 21 is not limited to glass, and it is sufficient if the material of the top plate 21 is optically transparent. Moreover, the top plate 21 may be surface-treated variously, and, for example, an antireflective coating may be formed over the top plate 21.

The image acquisition device 20 includes, in the housing, an image sensor 22, a plurality of light sources 23 that are arranged around the image sensor 22, and a temperature sensor 24. The image sensor 22 is, for example, a CCD (charge-coupled device) image sensor or a CMOS (complementary MOS) image sensor. The light source 23 is, for example, an LED (light emitting diode) light source. The temperature sensor 24 is a sensor that measures the temperature of the image acquisition device 20.

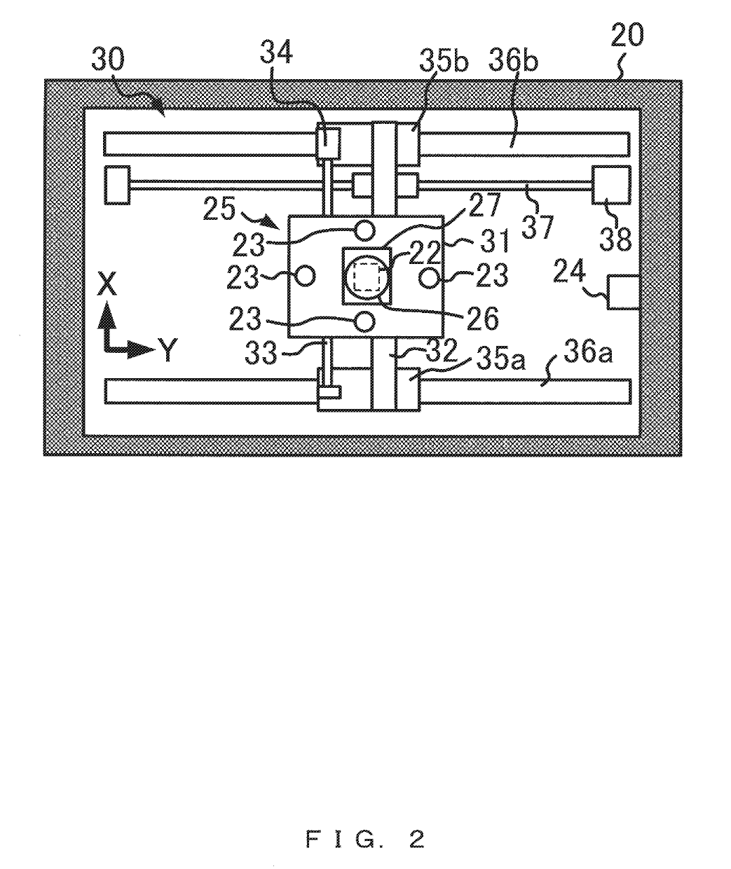

Next, the image acquisition device 20 is further described in detail with reference to FIGS. 2 to 4. FIG. 2 illustrates a configuration of the inside of the housing of the image acquisition device 20, and is a plan view of the image acquisition device 20 from which the top plate 21 has been removed. The image acquisition device 20 includes, in the housing, an image-capturing unit 25 that captures an image, a movement mechanism 30 that moves the image-capturing unit 25 within the housing, and the temperature sensor 24. The movement mechanism 30 is a mechanism that moves the image-capturing unit 25 two-dimensionally in a direction along the top plate 21.

The image-capturing unit 25 is provided on a slider 31 that constitutes the movement mechanism 30, and in addition to the image sensor 22 and the light source 23 described above, the image-capturing unit 25 includes a lens 26 that forms an optical image of a biological sample with light emitted from the biological sample, and a reference focus device 27 that moves the lens 26 in a direction of an optical axis of the lens 26.

The movement mechanism 30 includes the slider 31, a guiderail 32, a ball screw 33, and a motor 34 as a mechanism that moves the image-capturing unit 25 linearly in a certain direction along the top plate 21 (hereinafter referred to as an X direction). The slider 31 slides along the guiderail 32.

The motor 34 rotates following an instruction from the controller 40. The ball screw 33 is rotated due to the rotation of the motor 34 so that the slider 31 fixed to a nut combined with the ball screw 33 moves along the guiderail 32. Accordingly, the image-capturing unit 25 also moves in the X direction along the guiderail 32.

The movement mechanism 30 further includes a slider 35a and a slider 35b, a guiderail 36a and a guiderail 36b, a ball screw 37, and a motor 38 as a mechanism that moves the image-capturing unit 25 linearly in a direction (hereinafter referred to as a Y direction) along the top plate 21 that is perpendicular to the X direction. The slider 35a slides along the guiderail 36a. The slider 35b slides along the guiderail 36b. The guiderail 36a and the guiderail 36b are parallel to each other and oriented in a direction perpendicular to the guiderail 32.

The motor 38 rotates following an instruction from the controller 40. The ball screw 37 is rotated due to the rotation of the motor 38 so that the guiderail 32 fixed to a nut combined with the ball screw 37 moves. The guiderail 32 is fixed to the slider 35a and the slider 35b. Thus, the slider 35a and the slider 35b move together with the guiderail 32 along the guiderail 36a and the guiderail 36b. Accordingly, the image-capturing unit 25 also moves in the Y direction along the guiderail 36a and the guiderail 36b.

Following an instruction from the controller 40, the image acquisition device 20 rotates the motor 34 and the motor 38 that are power sources of the movement mechanism 30, so as to move the image-capturing unit 25 provided on the slider 31 to any position within the housing. Thus, even if a biological sample (or a monitoring target region) is larger than a field of view of the image acquisition device 20, it will be possible to monitor the entirety of the biological sample by acquiring images of the biological sample at a plurality of different image acquisition points. Here, the image acquisition point is a position of the image-capturing unit 25 at which the image acquisition device 20 acquires an image.

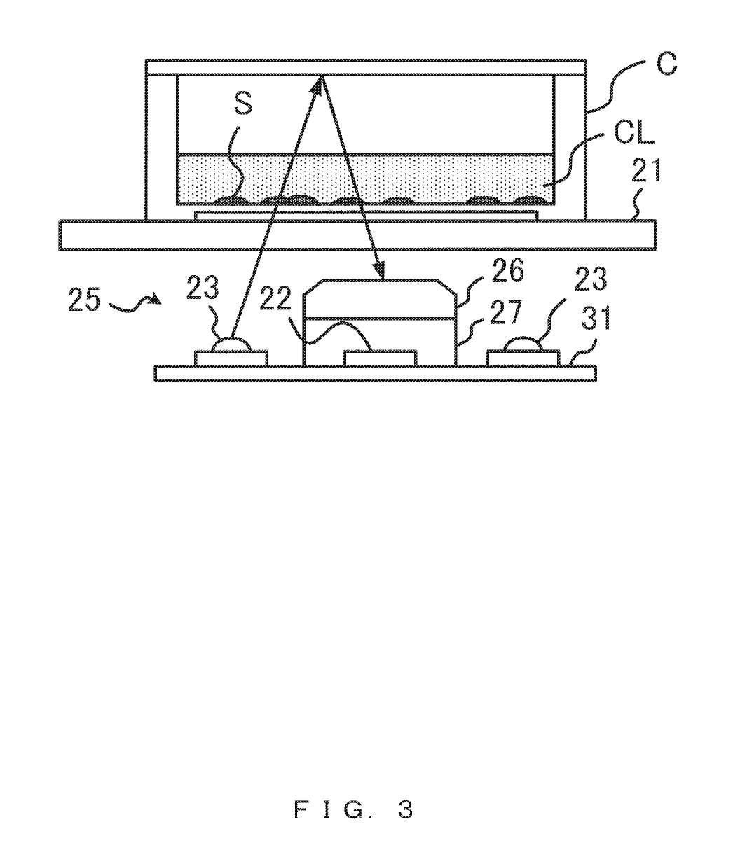

FIG. 3 is a diagram for explaining an image acquisition method performed by the image acquisition device 20. The image acquisition device 20 acquires an image of a biological sample following an instruction from the controller 40 after the image-capturing unit 25 is moved.

More specifically, at least one light source 23 from among of the plurality of light sources 23 arranged around the image sensor 22 emits light following an instruction from the controller 40. The determination of which of the plurality of light sources 23 emits light may be performed according to an image acquisition point. The light emitted from the at least one light source 23 enters the flask C passing through the top plate 21. The light is then reflected on an upper surface of the flask C and illuminates a biological sample S dipped into a culture CL from above. After that, the light that passed through the biological sample S enters the lens 26 through the top plate 21, and an optical image of the biological sample S is formed on the image sensor 22 due to an action of the lens 26. This results in generating an image signal in the image sensor 22.

The image acquisition device 20 controls the image-capturing unit 25 following an instruction from the controller 40, so as to acquire an image of a biological sample. In the image acquisition device 20, light that illuminates the biological sample obliquely enters the image sensor 22. This provides an effect similar to an Oblique illumination, which results in being able to acquire an image having a high contrast even if the biological sample is transparent.

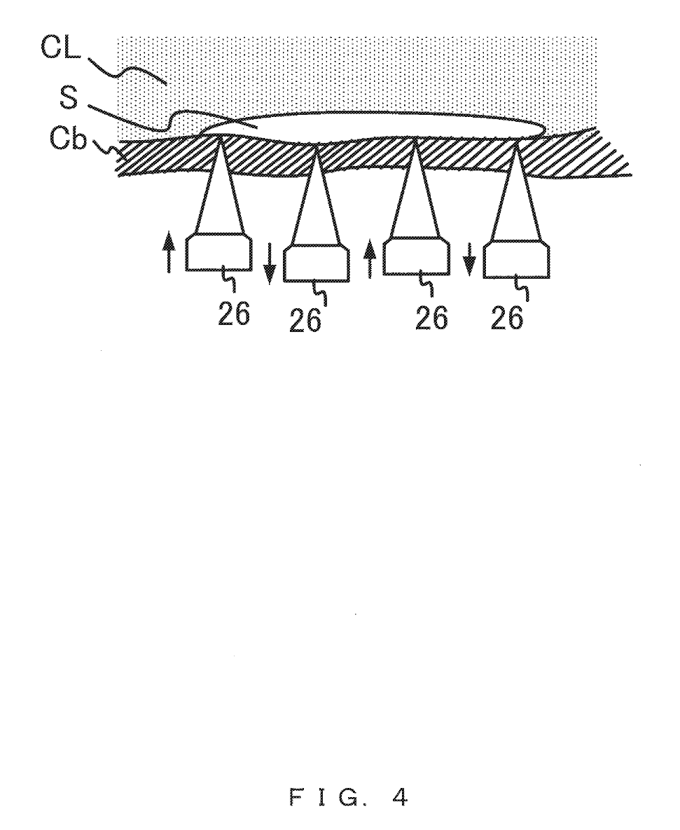

FIG. 4 is a diagram for explaining a focus operation performed by the image acquisition device 20 at the time of acquiring an image. The image acquisition device 20 performs autofocus (hereinafter referred to as AF) processing following an instruction from the controller 40 after the image-capturing unit 25 is moved and before an image is acquired.

The culture vessel such as the flask C is often made of, for example, plastic, and generally has a bottom surface Cb that has an ununiform thickness, as illustrated in FIG. 4. Thus, when the image-capturing unit 25 is moved, the biological sample S adherent to the bottom surface Cb may be out of focus. Thus, the image acquisition device 20 performs AF processing after the image-capturing unit 25 is moved, so that the biological sample S is in focus. The AF processing may be, for example, a contrast detection AF or a phase detection AF, although it is not limited to them.

Even if a culture vessel has a bottom surface Cb that has an ununiform thickness, the image acquisition device 20 can acquire a high-contrast image by performing the AF processing following an instruction from the controller 40 after the image-capturing unit 25 is moved.

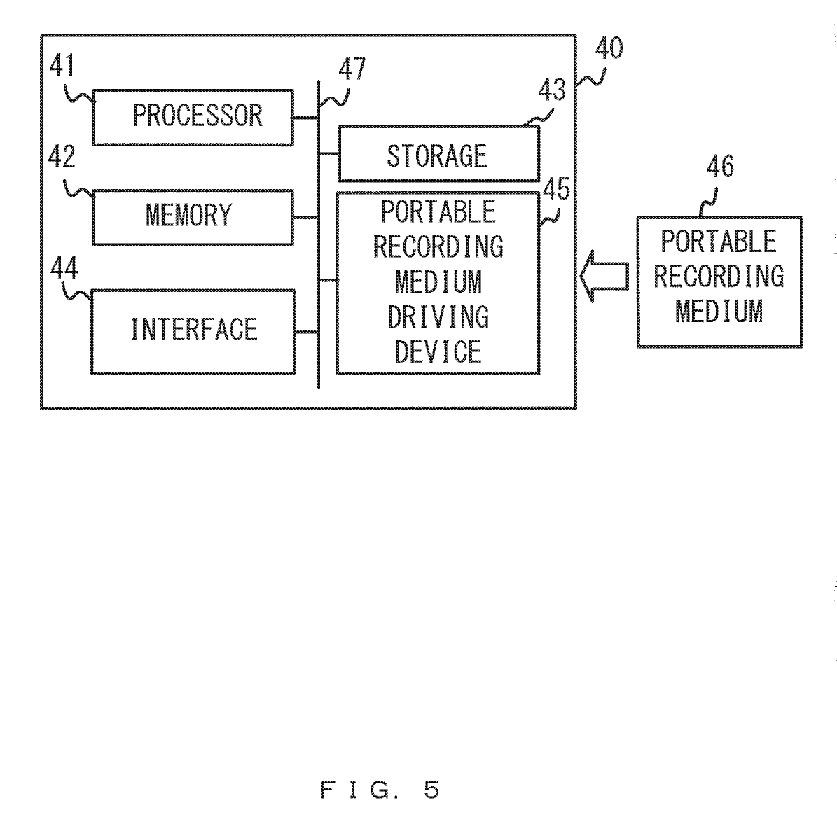

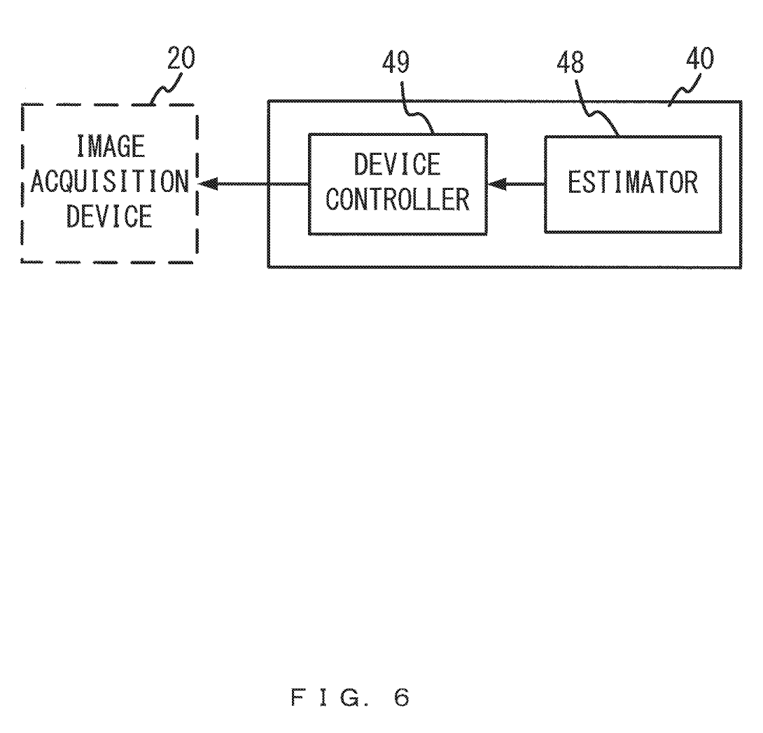

Next, the controller 40 is further described in detail with reference to FIGS. 5 and 6. FIG. 5 is a block diagram that illustrates a hardware configuration of the controller 40. The controller 40 is, for example, a standard computer. As illustrated in FIG. 5, the controller 40 includes a processor 41, a memory 42, a storage 43, an interface 44, and a portable recording medium driving device 45 into which a portable recording medium 46 is inserted, and these components are connected to one another through a bus 47.

The processor 41 is, for example, a CPU (central processing unit), an MPU (micro processing unit), or a DSP (digital signal processor), and performs programmed processing by executing a program. The memory 42 is, for example, a RAM (random access memory), and temporality stores therein a program or data stored in the storage 43 or the portable recording medium 46 upon the execution of a program.

The storage 43 is, for example, a hard disk or a flash memory, and is primarily used to store various data and programs. The interface 44 is, for example, a network card, and is a circuit that communicates a signal with a device other than the controller 40 (such as the image acquisition device 20, the liquid crystal display 50, the keyboard 60, the laptop computer 70, and the tablet computer 80).

The portable recording medium driving device 45 is used to accommodate the portable recording medium 46 such as an optical disk and CompactFlash®. The portable recording medium 46 plays a role in assisting the storage 43. Both the storage 43 and the portable recording medium 46 are examples of a non-transitory computer-readable recording medium that stores therein a program.

The configuration illustrated in FIG. 5 is an example of a hardware configuration of the controller 40, and the controller 40 is not limited to this configuration. The controller 40 may be a dedicated device, not a general-purpose device. The controller 40 may include an electric circuit such as an ASIC (application specific integrated circuit) or an FPGA (field programmable gate array) instead of or in addition to a processor that executes a program, and such an electric circuit may perform the entirety or a portion of processing described later.

FIG. 6 is a block diagram that illustrates a functional configuration of the controller 40. The controller 40 includes an estimator 48 that estimates a state of a temperature of a biological sample (hereinafter referred to as a temperature state) with respect to a set temperature of the incubator 10, and a device controller 49 that restricts an operation of the image acquisition device 20 at least according to the state estimated by the estimator 48. The processor 41 operates as the estimator 48 and the device controller 49 by executing a program.

For example, the estimator 48 estimates whether a temperature of a biological sample is in a state in which the temperature of the biological sample is higher than a set temperature with the difference in temperature being greater than or equal to a certain difference in temperature (hereinafter referred to as a high temperature state), or in a state other than the high temperature state (hereinafter referred to as a non-high-temperature state). The certain difference in temperature (hereinafter referred to as a first temperature difference) is a difference in temperature determined in advance, and is, for example, 1° C.

The first temperature difference may be determined from various viewpoints. It may be determined according to an acceptable temperature range for a culture environment, or according to the thermal tolerance of a biological sample. Further, when there is a possibility that an image quality will be degraded due to a change in temperature, the first temperature difference may be determined according to an acceptable level of degradation in image quality. For example, when the difference in temperature between the inside and the outside of the flask C becomes too large, the flask C may be fogged up. If an image of a biological sample is acquired through the fogged flask C, the contrast of the image will be decreased, which also results in a decrease in the accuracy in various measurements and analyses based on the acquired image. Thus, a difference in temperature with which a culture vessel is not fogged up may be determined to be the first temperature difference.

At least according to a state estimated by the estimator 48, the device controller 49 causes the image acquisition device 20 to transition from an operation state to a stopping state, the operation state being a state in which an image of a biological sample can be acquired, the stopping state being a state in which power consumption is lower than in the operation state. In the stopping state, power consumption is lower than in an idle state in which the image acquisition device 20 is waiting for an instruction from the controller 40. It is preferable that the image acquisition device 20 stop its communication function in the stopping state.

Specifically, for example, the device controller 49 transmits a stopping instruction indicating a stopping time period to the image acquisition device 20 at least according to the state estimated by the estimator 48. More specifically, for example, the device controller 49 transmits the stopping instruction indicating a stopping time period to the image acquisition device 20 at least when the device controller 49 estimates that the temperature is in a high temperature state.

It is sufficient if the stopping time period is a time period sufficient to decrease the temperature of a biological sample up to a set temperature from a temperature that is higher than a set temperature with the difference in temperature being the first temperature difference. The stopping time period may be, for example, a certain time period determined in advance, such as 15 minutes. Further, the stopping time period may be determined according to a continuous operation time until a stopping instruction is output, and it may be, for example, three times as long as the continuous operation time.

The image acquisition device 20 transitions from an operation state to a stopping state when the image acquisition device 20 receives a stopping instruction, and transitions from the stopping state to the operation state after a stopping time period indicated by the stopping instruction has elapsed. The image acquisition device 20 transitions from the stopping state to the operation state on its own initiative. In other words, the image acquisition device 20 transitions from the stopping state to the operation state independently, not by receiving an instruction from the controller 40. This permits the image acquisition device 20 to stop its communication function in the stopping state, so it is possible to further suppress power consumption.

According to the culture monitoring system 1 having the configuration described above, the controller 40 can estimate a temperature state of a biological sample so as to restrict an operation of the image acquisition device 20. This results in being able to suppress an increase in the temperature of the biological sample due to the heat generation of the image acquisition device 20.

The culture monitoring system 1 prevents the temperature of a biological sample from increasing excessively, comparing to when a cooling device is provided in the incubator 10 to cool the biological sample rapidly as needed. This provides the advantages of, for example, avoiding damaging a biological sample and preventing the biological sample from being cultured under a condition that is very different from the condition for an intended culture environment. Further, the culture monitoring system 1 can decrease the temperature of a biological sample gradually by restricting an operation of the image acquisition device 20. This prevents condensation that causes a significant degradation in image quality from forming, because the temperatures of the biological sample and a culture vessel (the flask C) are not changed rapidly.

Embodiments using the culture monitoring system 1 are specifically described below. The configuration of the culture monitoring system 1 according to each of the embodiments is as described above.

### First Embodiment

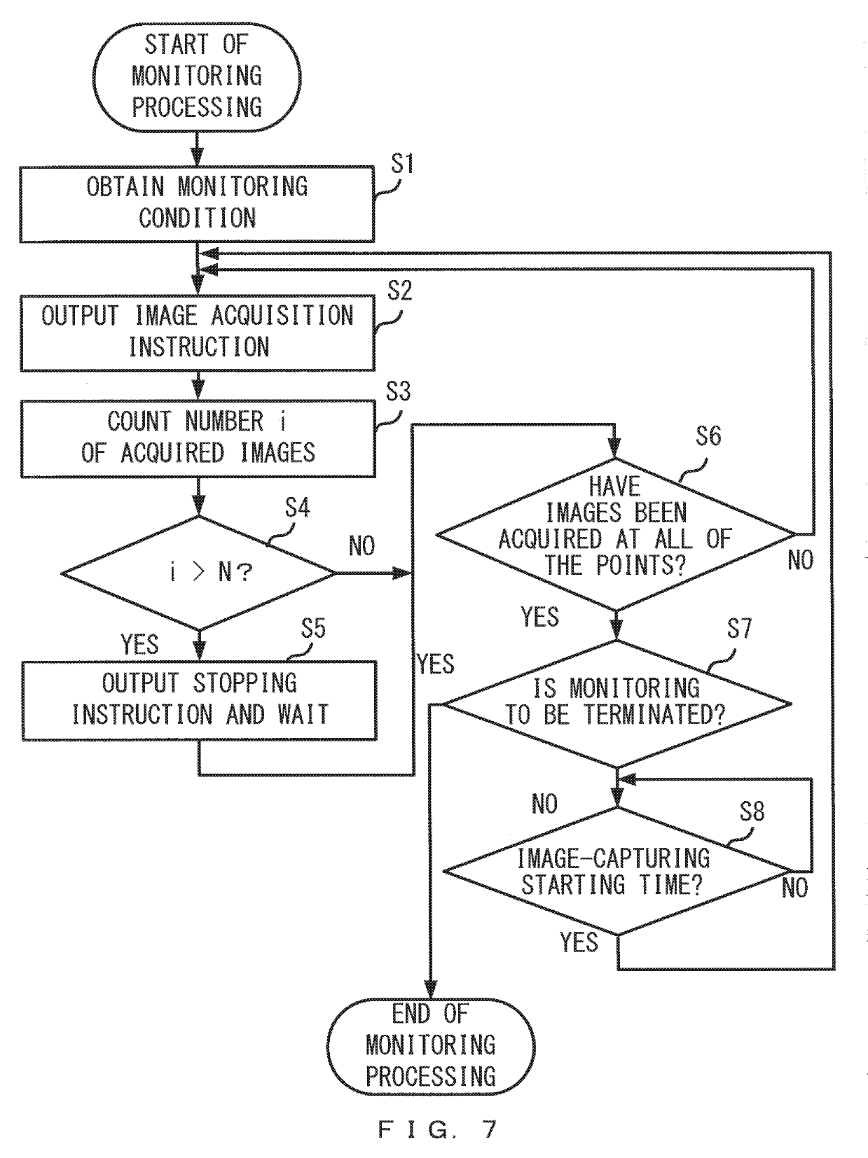

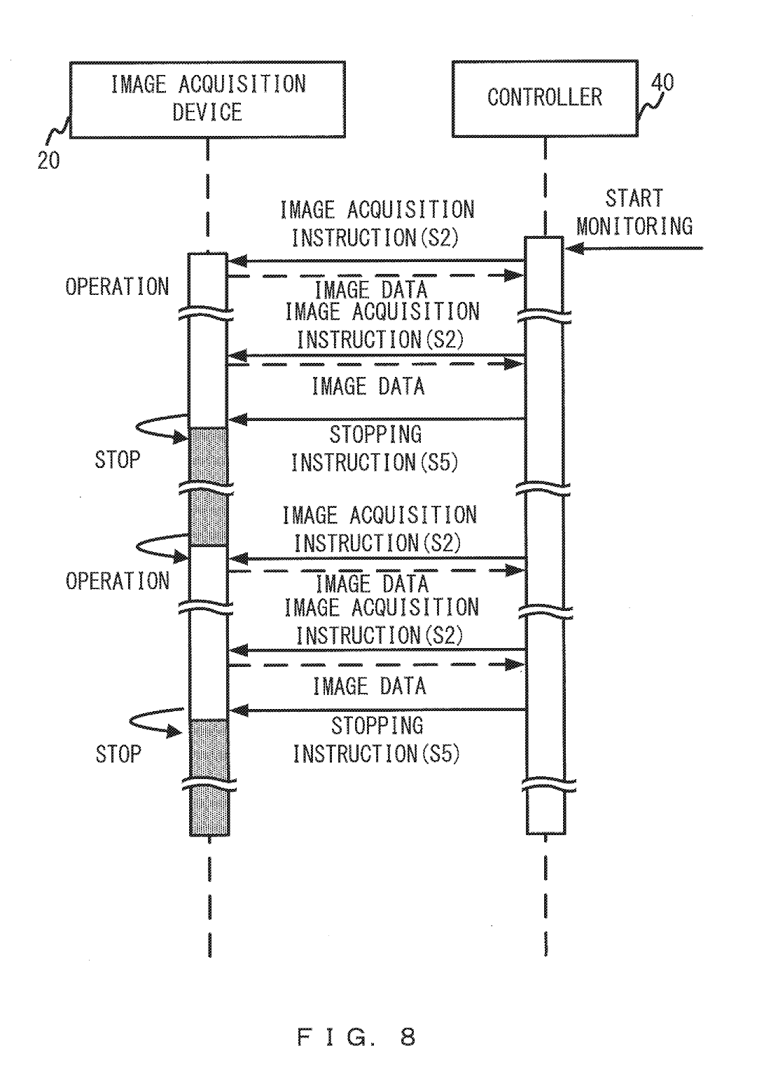

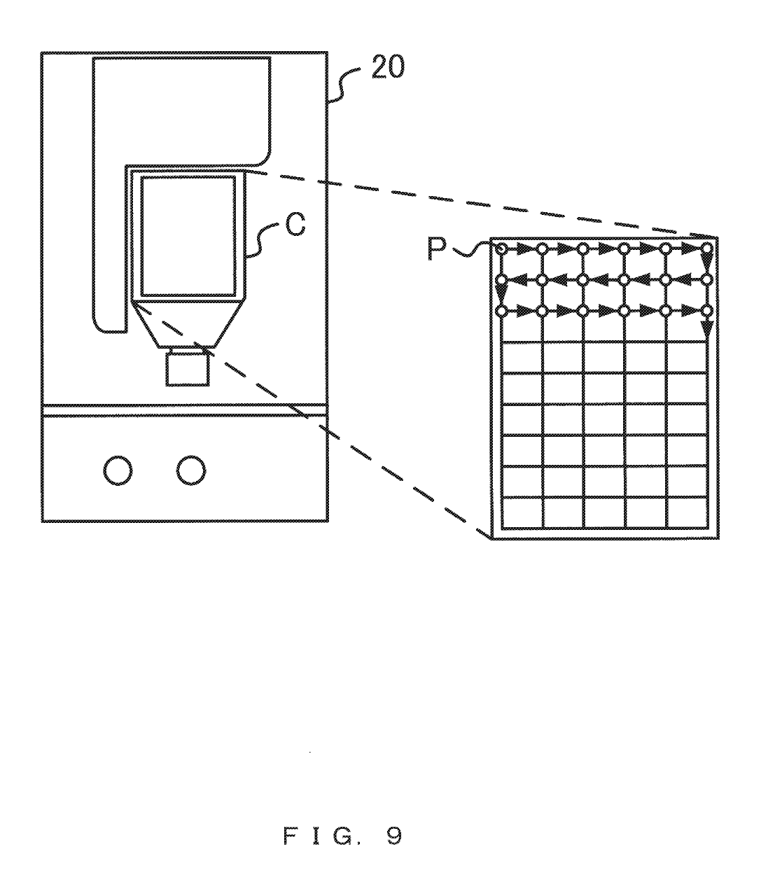

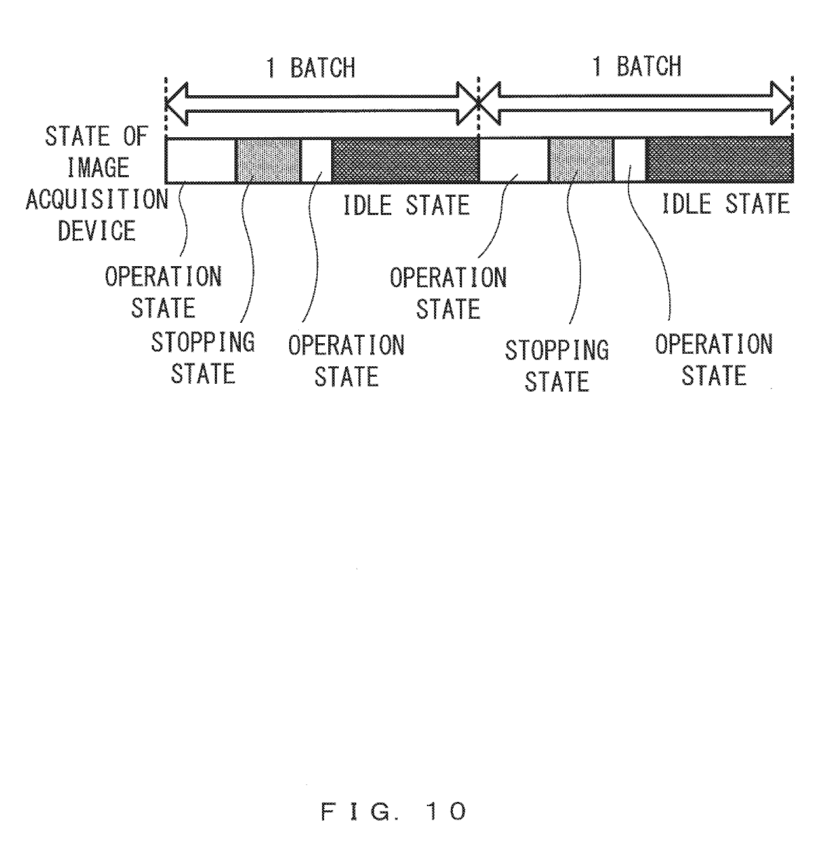

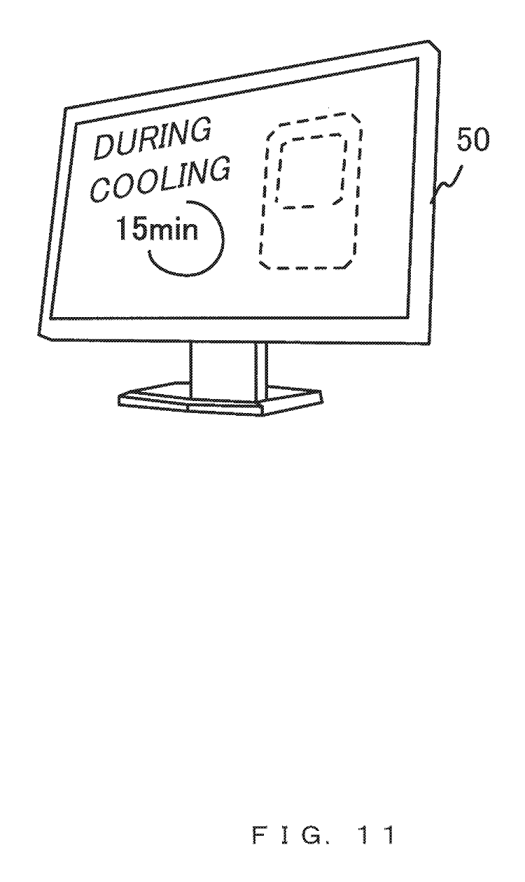

FIG. 7 is a flowchart that illustrates an example of monitoring processing according to the present embodiment. The monitoring processing illustrated in FIG. 7 is performed by the controller 40, and indicates a method for controlling the image acquisition device 20 arranged in the incubator 10. FIG. 8 is a sequence diagram that illustrates a communication of data between the controller 40 and the image acquisition device 20. FIG. 9 illustrates an image acquisition point according to the present embodiment. FIG. 10 illustrates an example of a change in a state of the image acquisition device 20 according to the present embodiment. FIG. 11 illustrates a screen displayed in a stopping state.

The culture monitoring system 1 according to the first embodiment is described below with reference to FIGS. 7 to 11 using, as an example, monitoring processing of monitoring a biological sample by performing time-lapse photography.

In the present embodiment, the estimator 48 estimates a temperature state on the basis of a history of the controller 40 controlling the image acquisition device 20. Specifically, the temperature state is estimated on the basis of the number of acquired images.

As illustrated in FIG. 8, when an monitoring starting instruction is input to the controller 40 using, for example, the keyboard 60, the controller 40 starts performing the monitoring processing illustrated in FIG. 7.

When the monitoring processing starts, first, the controller 40 obtains a monitoring condition (Step S1). Here, the controller 40 reads, for example, a monitoring condition recorded in the storage 43 in advance and stores the monitoring condition in the memory 42.

Examples of the monitoring condition are information on a position of at least one image acquisition point P (also simply referred to as a point), a stopping time period indicated by a stopping instruction, a threshold N for the number of acquired images, an image-capturing time interval between time-lapse photography and time-lapse photography, and the number of times of time-lapse photography. The threshold N is determined in advance with the criterion that the temperature state of a biological sample is changed to a high temperature state due to the heat generation of the image acquisition device 20 when the image acquisition device 20 acquires more than N images consecutively.

When the monitoring condition is obtained, the controller 40 outputs an image acquisition instruction to the image acquisition device 20 (Step S2). Here, as illustrated in FIG. 8, the controller 40 outputs, to the image acquisition device 20, an image acquisition instruction that includes information on a position of an image acquisition point P. The image acquisition instruction may include a plurality of instructions such as an instruction to move to an image acquisition point P, an autofocus instruction, and an image-capturing instruction.

When the image acquisition device 20 receives the image acquisition instruction, the image acquisition device 20 moves the image-capturing unit 25 to a specified image acquisition point P, as illustrated in FIG. 9, performs autofocus processing, and acquires an image. After that, the image acquisition device 20 outputs image data of the acquired image to the controller 40, as illustrated in FIG. 8.

When the controller 40 receives the image data from the image acquisition device 20, the controller 40 counts the number i of acquired images (Step S3). Here, on the basis of a control history, the controller 40 that includes the estimator 48 counts the number i of acquired images of a biological sample that are acquired by the image acquisition device 20. The number i of acquired images is the number of consecutively acquired images.

After that, the controller 40 determines whether the number i of acquired images is greater than the threshold N (Step S4). Taking into consideration the above-described criterion to determine the threshold N, it is assumed that the temperature state is a high temperature state when the number i of acquired images is greater than the threshold N, and the temperature state is a non-high-temperature state when the number i of acquired images is not greater than the threshold N. Thus, this is processing of estimating a temperature state on the basis of the counted number of acquired images.

When the number i of acquired images is not greater than the threshold N, the controller 40 determines whether images have been acquired at all of the image acquisition points P (Step S6). When the controller 40 has determined that all of the images have not been acquired, the controller 40 repeats the processes of Step S2 to Step S6. Accordingly, images of a biological sample are acquired sequentially at a plurality of image-capturing points, as illustrated in FIG. 9.

When the controller 40 has determined that the number i of acquired images is greater than the threshold N, the controller 40 outputs a stopping instruction to the image acquisition device 20 and waits for a stopping time period (Step S5). After that, the process of Step S6 is performed.

When the image acquisition device 20 receives the stopping instruction, the image acquisition device 20 transitions from an operation state to a stopping state, as illustrated in FIG. 8, so as to suppress power consumption. Then, the image acquisition device 20 returns to the operation state from the stopping state after the stopping time period indicated by the stopping instruction has elapsed.

When the controller 40 has determined that the images have been acquired at all of the image acquisition points P, the controller 40 determines whether monitoring is to be terminated (Step S7). Here, when time-lapse photography has not been performed the number of times of time-lapse photography that is obtained in Step S1, the controller 40 waits until the time of starting a next time-lapse photography (Step S8). After that, the processes of Step S2 to Step S8 are repeated. When time-lapse photography has been performed the obtained number of times of time-lapse photography, the controller 40 terminates the monitoring processing of FIG. 7.

In the present embodiment, when the image acquisition device 20 acquires more than N images consecutively during time-lapse photography performed regularly (1 batch), the image acquisition device 20 stops acquiring an image for a certain time period by transitioning from an operation state to a stopping state, and restarts acquiring an image by returning to the operation state, as illustrated in FIG. 10. This makes it possible to prevent the image acquisition device 20 from generating heat excessively. Thus, according to the culture monitoring system 1, it is possible to suppress an increase in the temperature of a biological sample due to the heat generation of the image acquisition device 20.

When the image acquisition device 20 is in the stopping state, the culture monitoring system 1 may display, on the liquid crystal display 50 that is a display device, a remaining time before the image acquisition device 20 returns to the operation state, for example, as illustrated in FIG. 11. The display of the remaining time permits a user to know the state of the image acquisition device 20, and this results in being able to remove anxieties of a user who is worried about a failure in the image acquisition device 20, the anxieties may occurring when the image acquisition device 20 stops suddenly.

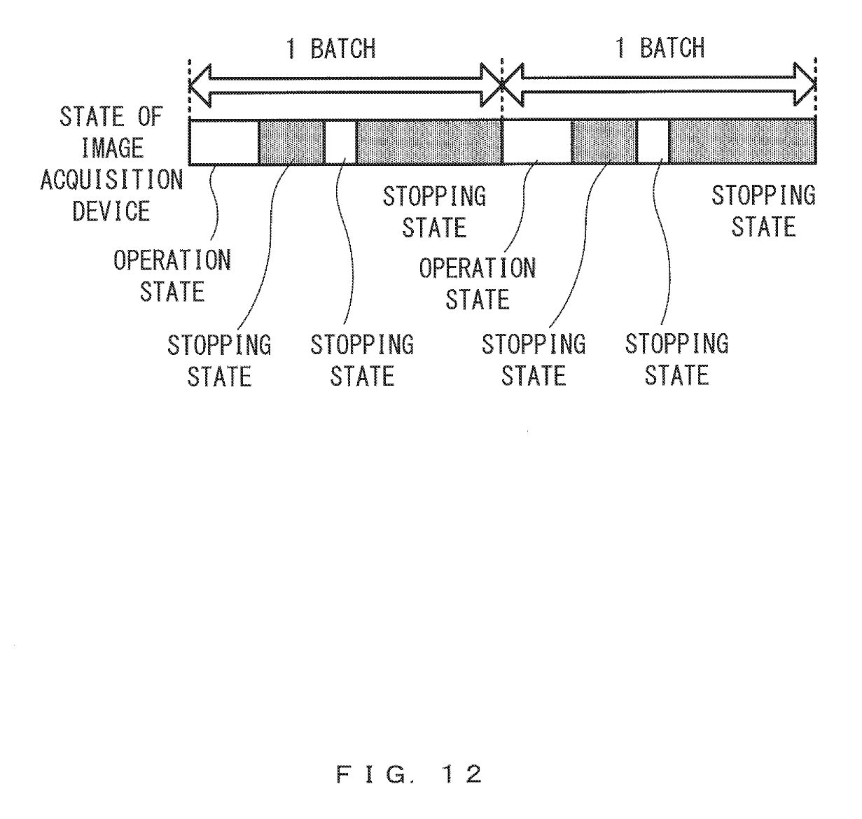

The example in which the stopping time period is constant has been described, but the stopping time period indicated by a stopping instruction is not limited to a certain time period, and it is sufficient if it is a time period sufficient to decrease the temperature of a biological sample up to a set temperature. For example, the example in which the image acquisition device 20 transitions to an idle state (an instruction waiting state) after images are acquired at all of the image acquisition points has been described in FIG. 10, but the image acquisition device 20 may transition to the stopping state as illustrated in FIG. 12. In this case, the controller 40 may specify, as a stopping time period, a time period from the end of an operation state to the start of a next time-lapse photography (a next butch) and may output the stopping instruction to the image acquisition device 20.

### Second Embodiment

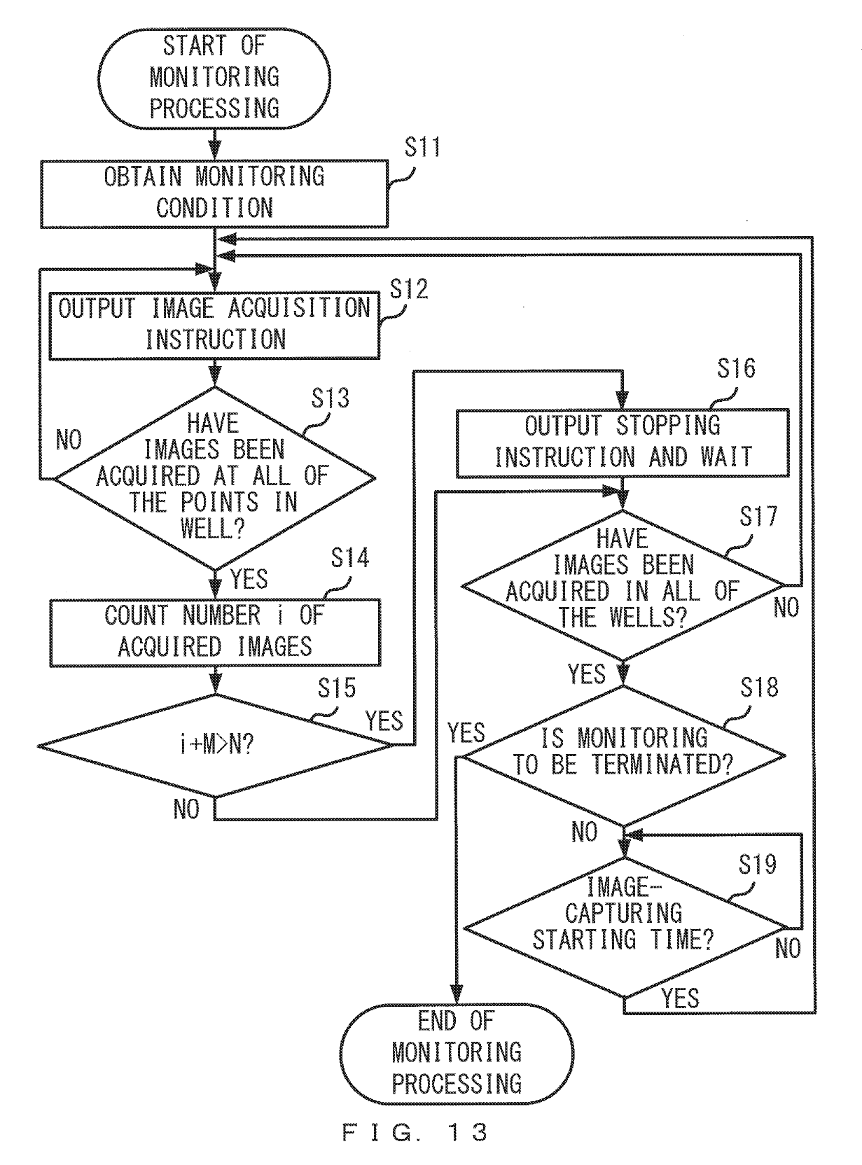

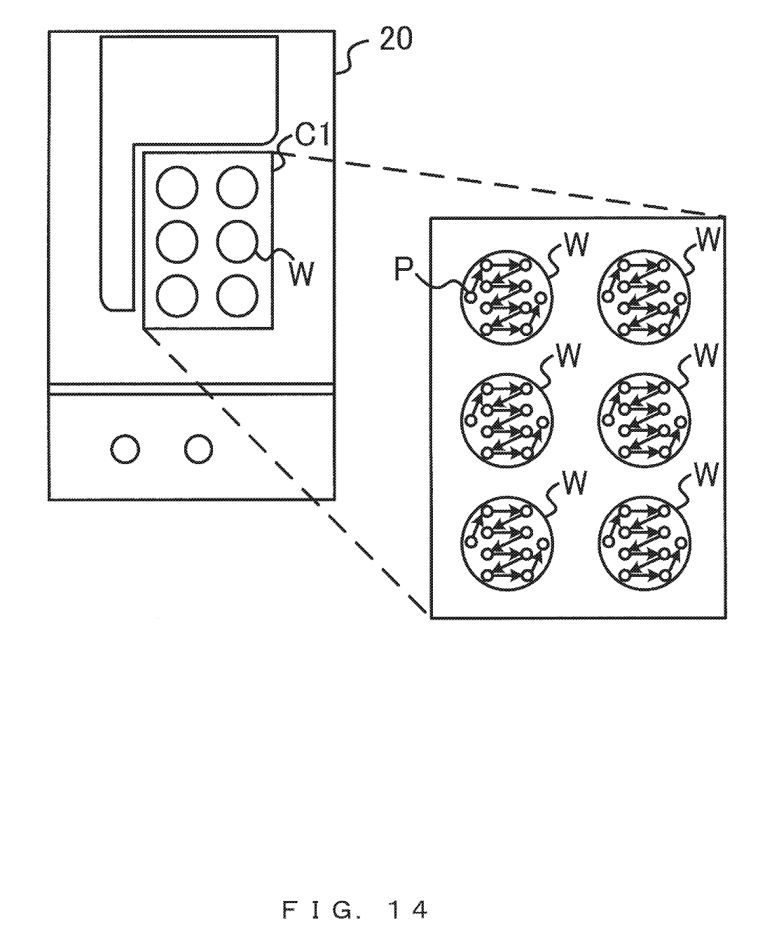

FIG. 13 is a flowchart that illustrates an example of monitoring processing according to the present embodiment. FIG. 14 illustrates an image acquisition point according to the present embodiment.

The culture monitoring system 1 according to the second embodiment is described below with reference to FIGS. 13 and 14 using, as an example, the monitoring processing of monitoring a biological sample by performing time-lapse photography. The present embodiment is different from the first embodiment in that, instead of the flask C, a multi-well plate C1 is used as a culture vessel, and in that a biological sample is accommodated in each of a plurality of wells W formed on the multi-well plate C1, as illustrated in FIG. 14. It is also different from the first embodiment in that monitoring processing illustrated in FIG. 13 is performed instead of the monitoring processing illustrated in FIG. 7.

In the present embodiment, a plurality of biological samples accommodated in a plurality of regions (the plurality of wells W) obtained by partitioning a culture vessel are monitoring targets. The device controller 49 restricts an operation of the image acquisition device 20 at least according to a temperature state and the number of image acquisition points, the number of image acquisition points being set in each of the plurality of regions.

When a monitoring starting instruction is input to the controller 40 using, for example, the keyboard 60, the controller 40 starts performing the monitoring processing illustrated in FIG. 13.

When the monitoring processing starts, first, the controller 40 obtains a monitoring condition (Step S11). Then, the controller 40 outputs an image acquisition instruction to the image acquisition device 20 (Step S12). These processes are similar to the processes of Step S1 and Step S2 of FIG. 7.

Next, the controller 40 determines whether images have been acquired at all of the image acquisition points P in a currently targeted well (Step S13). When all of the images have not been acquired, the controller 40 repeats the processes of Step S12 and Step S13.

When the images have been acquired at all of the image acquisition points P in the currently targeted well, the controller 40 counts the number i of acquired images (Step S14). This process is similar to the process of Step S3 of FIG. 7. In other words, on the basis of a control history, the controller 40 counts the number i of acquired images of a biological sample that are acquired by the image acquisition device 20.

After that, the controller 40 determines whether a value obtained by adding the number M of image acquisition points that is set for a next well to the number i of acquired images that is counted in Step S14 is greater than a threshold N (Step S15), the next well being a well in which a biological sample whose image is to be acquired next is accommodated.

When the value obtained by adding the number i of acquired images and the number M of image acquisition points is not greater than the threshold N, the controller 40 determines whether images have been acquired in all of the wells in the multi-well plate C1 (Step S17). When the controller 40 has determined that all of the images have not been acquired, the controller 40 repeats the processes of Step S12 to Step S17 for a next well.

When the value obtained by adding the number i of acquired images and the number M of image acquisition points is greater than the threshold N, the controller 40 outputs a stopping instruction to the image acquisition device 20 and waits for a stopping time period (Step S16). After that, the process of Step S17 is performed.

When the controller 40 has determined that the images have been acquired in all of the wells specified as monitoring targets, the controller 40 determines whether monitoring is to be terminated (Step S18). This process is similar to the process of Step S7 of FIG. 7.

When the controller 40 has determined that the monitoring is not to be terminated, the controller 40 waits until the time of starting a next time-lapse photography (Step S19). After that, the processes of Step S12 to Step S19 are repeated until it is determined that the monitoring is to be terminated.

As in the case of the first embodiment, the culture monitoring system 1 according to the present embodiment also makes it possible to suppress an increase in the temperature of a biological sample due to the heat generation of the image acquisition device 20.

Further, in the present embodiment, before the controller 40 starts acquiring an image of a biological sample accommodated in a next well W, the controller 40 determines whether the number of acquired images will exceed N in the process of acquiring images consecutively at a plurality of image acquisition points P set in a next well W. When the controller 40 has determined that the number of acquired images will exceed N, the controller 40 outputs a stopping instruction to the image acquisition device 20 before the controller 40 starts acquiring an image of a biological sample accommodated in the next well W. This prevents the image acquisition device 20 from transitioning to a stopping state before images are acquired at all of the image acquisition points in a well W (in the process of acquiring an image for a well).

### Third Embodiment

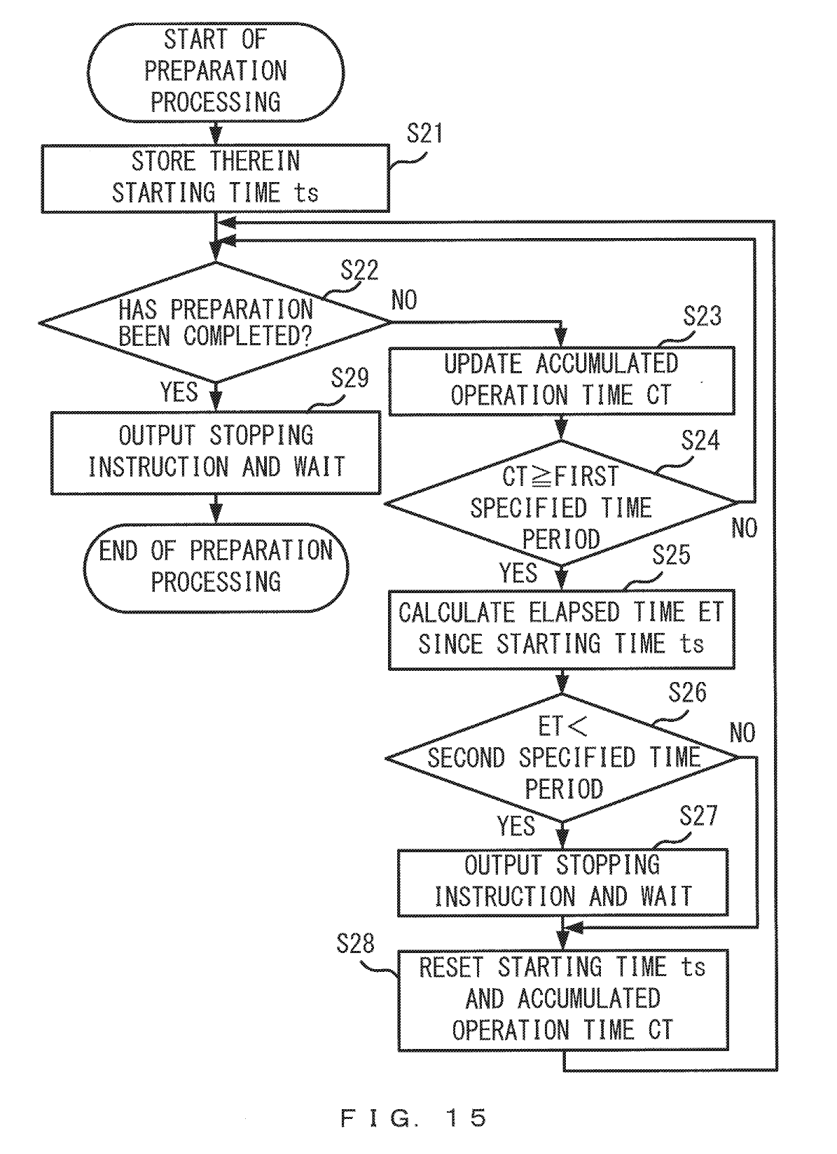

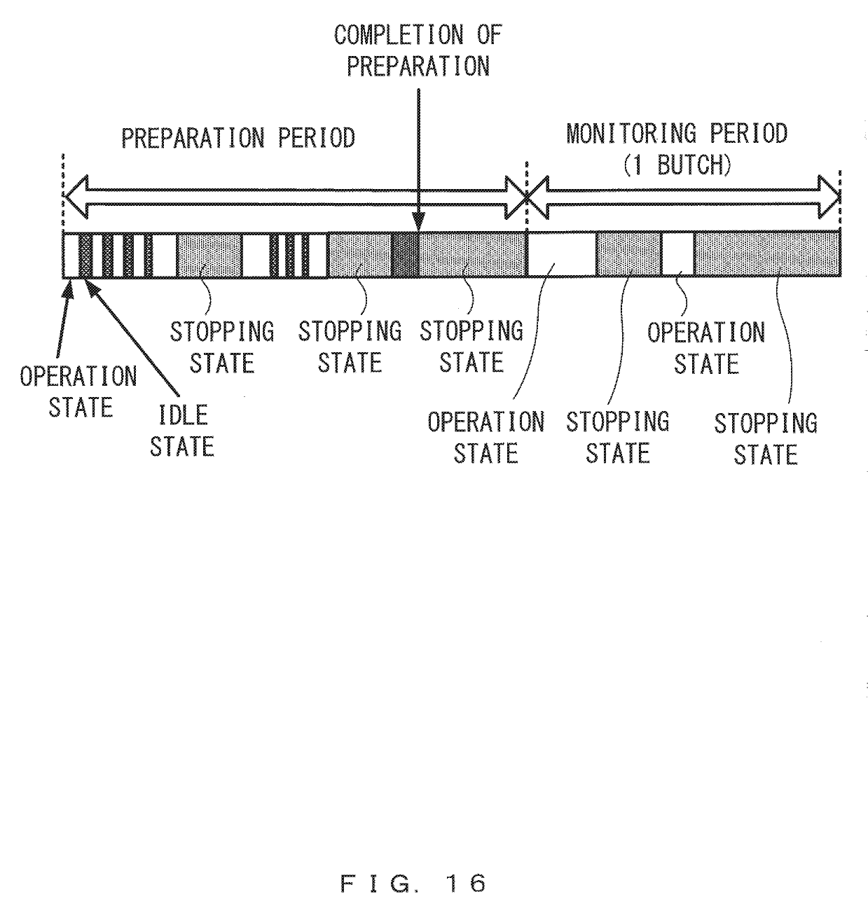

FIG. 15 is a flowchart that illustrates preparation processing according to the present embodiment. FIG. 16 illustrates an example of a change in a state of the image acquisition device 20 according to the present embodiment.

The culture monitoring system 1 according to the third embodiment is described below with reference to FIGS. 15 and 16 using, as an example, the preparation processing in which a user manipulates the image acquisition device 20 manually before monitoring processing is started. The preparation processing is performed in order to, for example, adjust a condition for acquiring an image or specify a plurality of image acquisition points. Here, the image acquisition device 20 performs various operations for an image acquisition according to an arbitrary manual manipulation performed by a user.

In the present embodiment, during a preparation period in which the preparation processing is performed, the estimator 48 determines, on the basis of a control history, whether an accumulated operation time obtained by accumulating operation times of the image acquisition device 20 reached a first specified time period before a second specified time period has elapsed since a reference time. Then, the estimator 48 estimates a temperature state on the basis of a result of the determination.

When the preparation processing is started, first, the controller 40 stores therein a starting time is as a reference time (Step S21). Then, the controller 40 determines whether preparation has been completed (Step S22). For example, when a completion instruction has been input using an input device, the controller 40 has determined that the preparation has been completed.

When the controller 40 has determined that the preparation has been completed, the controller 40 outputs a stopping instruction to the image acquisition device 20 and waits for a stopping time period (Step S29). After that, the preparation processing is terminated.

When the controller 40 has determined that the preparation has not been completed, the controller 40 updates an accumulated operation time CT (Step S23). Here, the controller 40 calculates the accumulated operation time CT on the basis of a history of the controller 40 controlling the image acquisition device 20.

The accumulated operation time is a total time period for which the image acquisition device 20 actually operated in accordance with a manipulation of a user. In other words, the accumulated operation time is a total time period for which the image acquisition device 20 is in an operation state in accordance with a manipulation of a user, and does not include a time period for which the image acquisition device 20 is in an idle state. For example, when a user successively gives instructions of a manual focus movement, a live image display, a movement of the image-capturing unit 25, an autofocus, and a still image acquisition, the accumulated operation time is a total time period obtained by adding time periods for which the image acquisition device 20 operates in accordance with the respective instructions.

Next, the controller 40 determines whether the accumulated operation time CT has reached a first specified time period (Step S24). When the controller 40 has determined that the accumulated operation time CT has not reached the first specified time period, the controller 40 repeats the processes of Step S22 to Step S24.

The first specified time period is a time period determined in advance, and is, for example, ten minutes. It is sufficient if the first specified time period is a time period that satisfies the following: the temperature of a biological sample is expected to reach a certain temperature when the image acquisition device 20 operates for the first specified time period, the certain temperature being higher than a set temperature with the difference in temperature being a first temperature difference. For example, a time needed to consecutively acquire the number of images that is equal to the threshold N used when the monitoring processing has been described above may be measured in advance, and the measured time may be determined to be the first specified time period.

When the controller 40 has determined that the accumulated operation time CT has reached the first specified time period, the controller 40 calculates an elapsed time ET since the starting time ts (Step S25). The elapsed time ET corresponds to a time difference between a time at which the first specified time period was reached and the starting time ts.

When the elapsed time ET has been calculated, the controller 40 determines whether the elapsed time ET is less than the second specified time period (Step S26). The second specified time period is longer than the first specified time period and is, for example, 40 minutes.

When the controller 40 has determined that the elapsed time ET is less than the second specified time period, the controller 40 outputs a stopping instruction to the image acquisition device 20 and waits for a stopping time period (Step S27). Then, the controller 40 resets the starting time ts and the accumulated operation time CT (Step S28), and the processes of Step S22 to Step S28 are repeated until it is determined that the preparation has been completed.

When the controller 40 has determined that the elapsed time ET is not less than the second specified time period, the controller 40 resets the starting time ts and the accumulated operation time CT without outputting the stopping instruction (Step S28). The reason is that, when greater than or equal to the second specified time period has elapsed since the starting time, it is expected that an increase in the temperature of a biological sample will hardly occur even if the image acquisition device 20 operates for the first specified time period in total.

When the controller 40 resets the starting time ts and the accumulated operation time CT, the controller 40 repeats the processes of Step S22 to Step S28 until it is determined that the preparation has been completed.

In the present embodiment, the image acquisition device 20 transitions from an operation state to a stopping state when the image acquisition device 20 operates for greater than or equal to a certain accumulated time period (the first specified time period) during a preparation period in which the image acquisition device 20 operates intermittently in accordance with a manipulation of a user, as illustrated in FIG. 16. This makes it possible to prevent the image acquisition device 20 from generating heat excessively. Thus, the culture monitoring system 1 according to the present embodiment also makes it possible to suppress an increase in the temperature of a biological sample due to the heat generation of the image acquisition device 20.

Further, when it took a long time for the image acquisition device 20 to operate for a certain accumulated time period, the transition to a stopping state will be omitted even if the image acquisition device 20 operated for greater than or equal to the certain accumulated time period. This prevents the image acquisition device 20 from stopping needlessly in spite of no increase in the temperature of a biological sample.

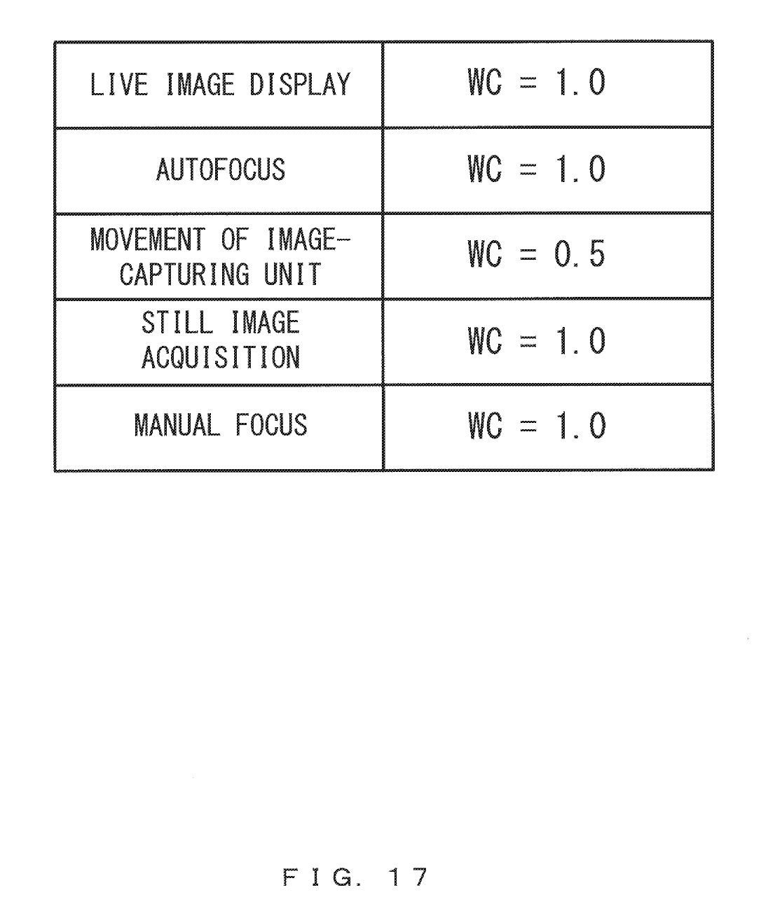

In the present embodiment, the example in which the estimator 48 determines whether an accumulated operation time has reached the first specified time period has been described, but instead of the accumulated operation time, the estimator 48 may calculate an accumulated operation time obtained by accumulating operation times that have been weighted according to power consumption (hereinafter referred to as a weighted accumulated operation time). The weighted accumulated operation time may be calculated using a weighting coefficient list, for example, as illustrated in FIG. 17, in which a weighting coefficient WC is listed for each processing performed in the image acquisition device 20. FIG. 17 illustrates an example in which less power is consumed and the weighting coefficient WC is smaller at the time of performing processing of the movement of the image-capturing unit 25, compared to the other processing performed in the image acquisition device 20.

When a weighted accumulated operation time is calculated, the estimator 48 may determine, on the basis of a control history, whether the weighted accumulated operation time obtained by accumulating operation times of the image acquisition device 20 that have been weighted according to power consumption reached a first specified time period before a second specified time period has elapsed since a reference time, so as to estimate a temperature state on the basis of a result of the determination of whether the first specified time period was reached. This makes it possible to determine, with a higher degree of accuracy, whether there is a need for a transition to a stopping state, which results in being able to suppress an increase in the temperature of a biological sample and to prevent the image acquisition device 20 from stopping needlessly.

### Fourth Embodiment

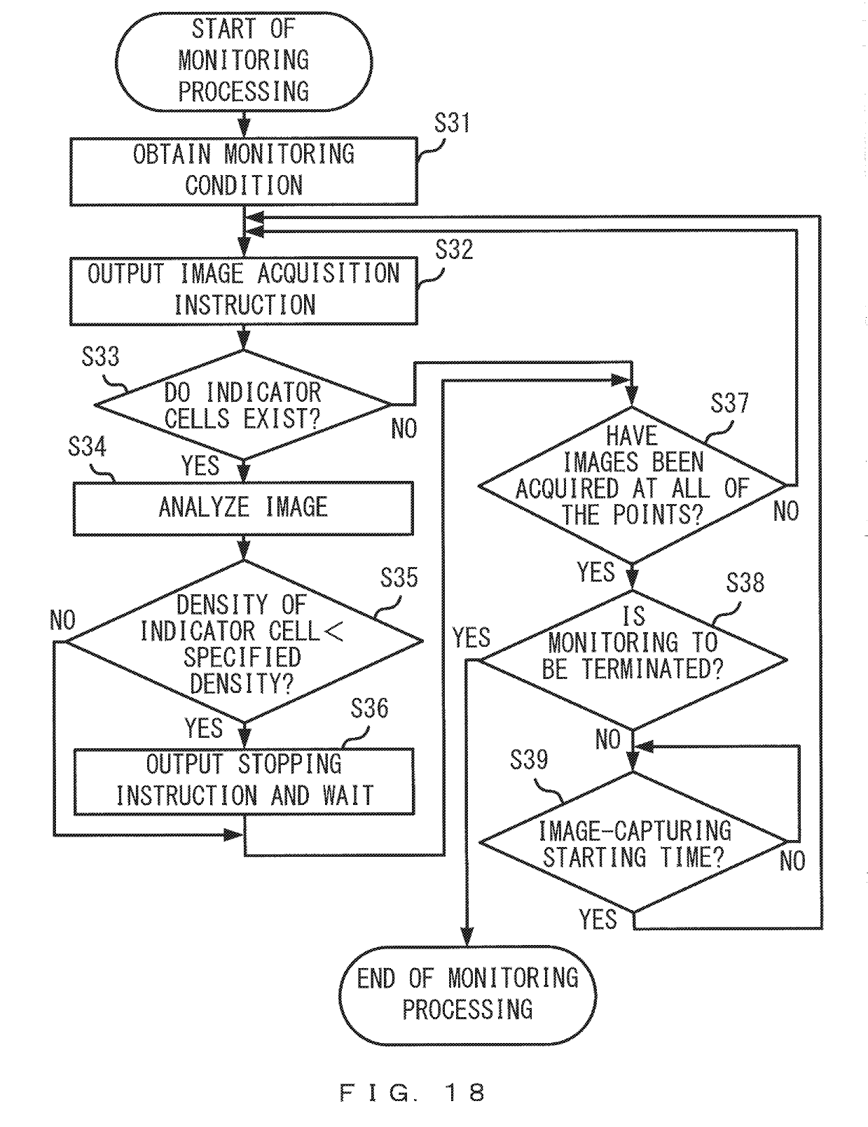

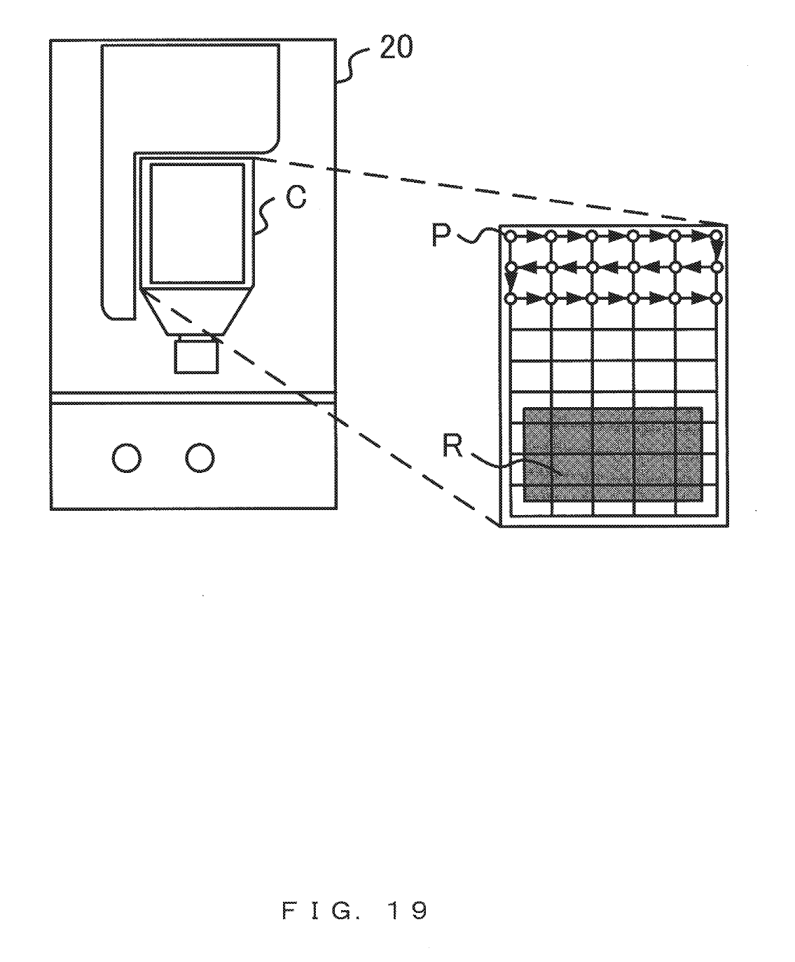

FIG. 18 is a flowchart that illustrates an example of monitoring processing according to the present embodiment. FIG. 19 illustrates an image acquisition point according to the present embodiment.

The culture monitoring system 1 according to the fourth embodiment is described below with reference to FIGS. 18 and 19 using, as an example, the monitoring processing of monitoring a biological sample by performing time-lapse photography. The present embodiment is different from the first embodiment in that monitoring processing illustrated in FIG. 18 is performed instead of the monitoring processing illustrated in FIG. 7 and in that a region R, in the flask C, in which indicator cells exist in a confluent state is known. Here, the indicator cell is a cell whose density varies depending on a temperature.

In the present embodiment, the estimator 48 estimates a temperature state on the basis of an image acquired by the image acquisition device 20. Specifically, the estimator 48 estimates the temperature state on the basis of an image of a biological sample that is acquired by the image acquisition device 20. More specifically, the estimator 48 estimates the temperature state on the basis of an image of the region R in which indicator cells of the biological sample exist in a confluent state.

When a monitoring starting instruction is input to the controller 40 using, for example, the keyboard 60, the controller 40 starts performing the monitoring processing illustrated in FIG. 18.

When the monitoring processing starts, first, the controller 40 obtains a monitoring condition (Step S31). Then, the controller 40 outputs an image acquisition instruction to the image acquisition device 20 (Step S32). These processes are similar to the processes of Step S1 and Step S2 of FIG. 7.

Next, the controller 40 determines whether indicator cells are included in an acquired image (Step S33). Here, the determination may be performed by determining whether an image acquisition point is within the range R illustrated in FIG. 19.

When the controller 40 has determined that the indicator cells are included, the controller 40 analyzes the acquired image (Step S34) and determines whether a density of the indicator cells that is obtained by a result of the analysis is less than a specified density (Step S35).

When the density of the indicator cells is less than the specified density, the controller 40 outputs a stopping instruction to the image acquisition device 20 and waits for a stopping time period, because it is expected that the temperature of the biological sample has been increased excessively (Step S36). Then, the controller 40 determines whether images have been acquired at all of the image acquisition points P (Step S37).

When the density of the indicator cells is not less than the specified density, the controller 40 determines whether images have been acquired at all of the image acquisition points P without outputting the stopping instruction (Step S37). Further, when the controller 40 has determined in Step S33 that the indicator cells are not included, the controller 40 also determines whether the images have been acquired at all of the image acquisition points P (Step S37). The processes of and after Step S37 are similar to the processes of Step S6 to Step S8 of FIG. 7.

As in the case of the first embodiment, the culture monitoring system 1 according to the present embodiment also makes it possible to suppress an increase in the temperature of a biological sample due to the heat generation of the image acquisition device 20.

### Fifth Embodiment

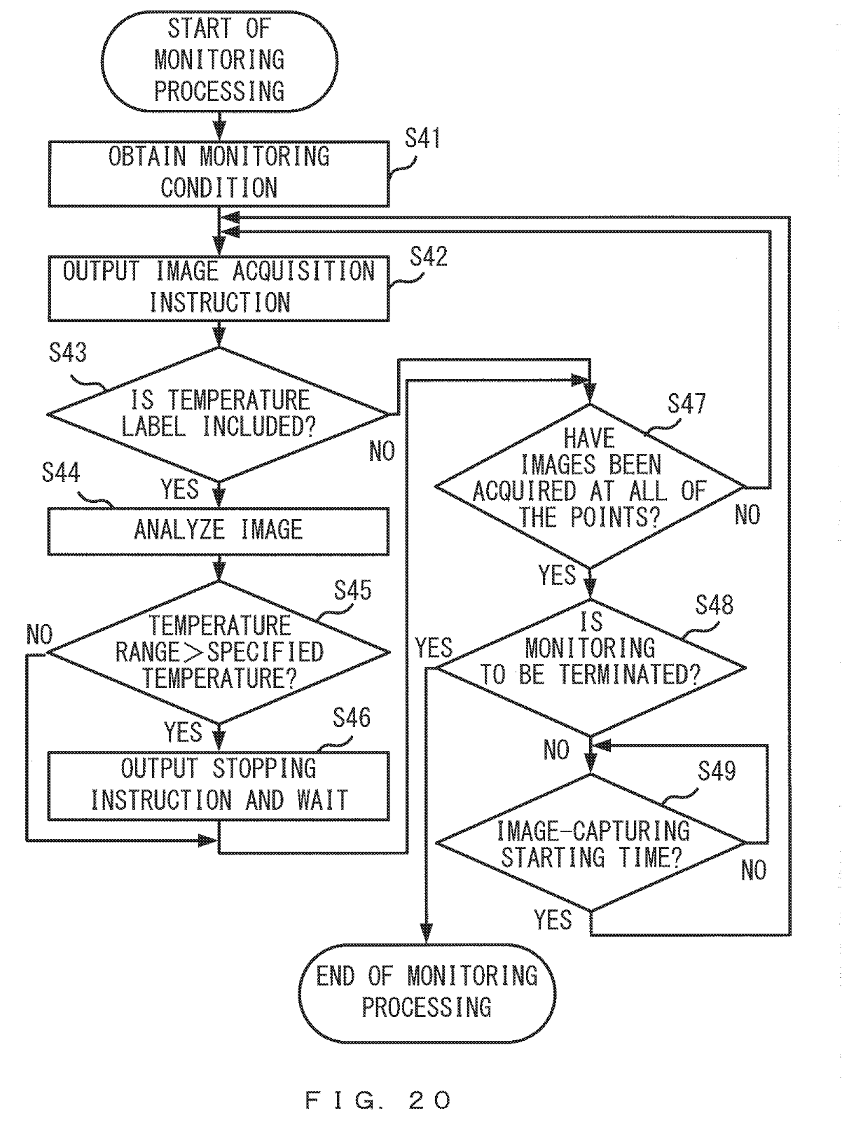

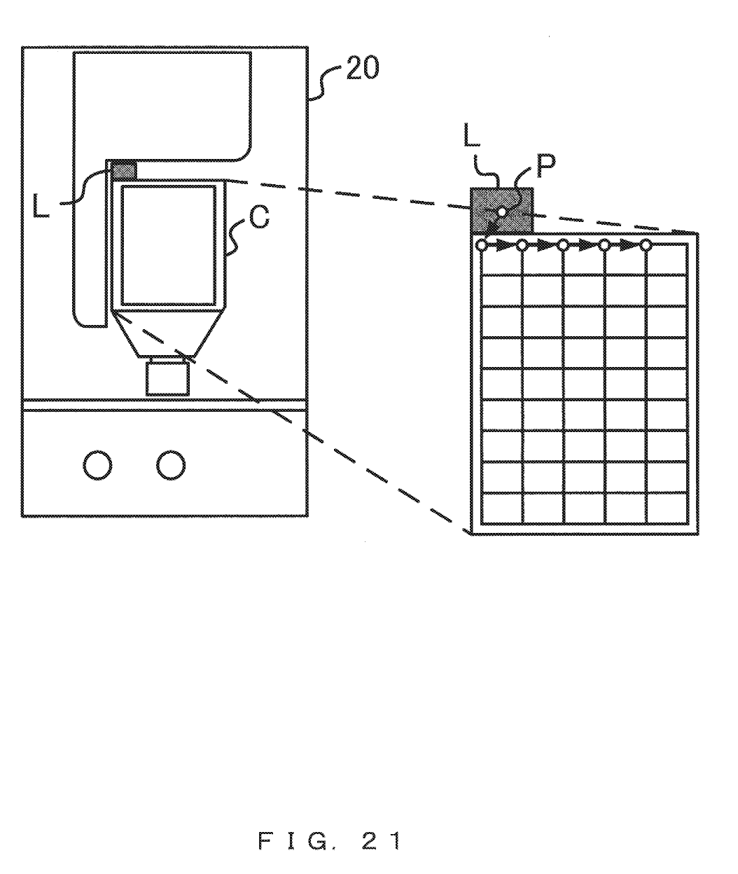

FIG. 20 is a flowchart that illustrates an example of monitoring processing according to the present embodiment. FIG. 21 illustrates an image acquisition point according to the present embodiment.

The culture monitoring system 1 according to the fifth embodiment is described below with reference to FIGS. 20 and 21 using, as an example, the monitoring processing of monitoring a biological sample by performing time-lapse photography. The present embodiment is different from the first embodiment in that monitoring processing illustrated in FIG. 20 is performed instead of the monitoring processing illustrated in FIG. 7. Further, it is also different from the first embodiment in that the image acquisition device 20 includes a temperature label L that is a member whose color is changed according to a temperature, and acquires an image of the temperature label L in addition to an image of a biological sample. Here, the temperature label L is a temperature label in which the relationship between a color of the temperature label L and a temperature range is known.

In the present embodiment, the estimator 48 estimates a temperature state on the basis of an image acquired by the image acquisition device 20. Specifically, the estimator 48 estimates the temperature state on the basis of an image of a temperature label L that is acquired by the image acquisition device 20.

When a monitoring starting instruction is input to the controller 40 using, for example, the keyboard 60, the controller 40 starts performing the monitoring processing illustrated in FIG. 20.

When the monitoring processing starts, first, the controller 40 obtains a monitoring condition (Step S41). Then, the controller 40 outputs an image acquisition instruction to the image acquisition device 20 (Step S42). These processes are similar to the processes of Step S1 and Step S2 of FIG. 7.

Next, the controller 40 determines whether a temperature label L is included in an acquired image (Step S43). Here, the determination may be performed by determining whether an image acquisition point is positioned in the label L illustrated in FIG. 21.

When the controller 40 has determined that the temperature label L is included, the controller 40 analyzes the acquired image (Step S44) and specifies a temperature range from a color of the temperature label L that is obtained by a result of the analysis. Then, the controller 40 determines whether the specified temperature range exceeds a specified temperature (Step S45).

When the specified temperature range exceeds the specified temperature, the controller 40 outputs a stopping instruction to the image acquisition device 20 and waits for a stopping time period, because it is expected that the temperature of the biological sample has been increased excessively (Step S46). Then, the controller 40 determines whether images have been acquired at all of the image acquisition points P (Step S47).

When the specified temperature range does not exceed the specified temperature, the controller 40 determines whether images have been acquired at all of the image acquisition points P without outputting the stopping instruction (Step S47). Further, when the controller 40 has determined in Step S43 that the temperature label L is not included, the controller 40 also determines whether the images have been acquired at all of the image acquisition points P (Step S47). The processes of and after Step S47 are similar to the processes of Step S6 to Step S8 of FIG. 7.

As in the case of the first embodiment, the culture monitoring system 1 according to the present embodiment also makes it possible to suppress an increase in the temperature of a biological sample due to the heat generation of the image acquisition device 20.

The embodiments described above are just examples to facilitate understanding of the present invention, and the embodiment of the present invention is not limited to these examples. Various modifications and alterations may be made to the culture monitoring system, the control method, and the program without departing from the scope of the invention specified in the claims.

In the embodiments described above, the example in which the estimator 48 estimates a temperature state on the basis of a history of controlling the image acquisition device 20 and on the basis of an image acquired by the image acquisition device 20 has been described, but the temperature state of a biological sample may be estimated on the basis of a temperature measured by the temperature sensor 24 included in the image acquisition device 20.

In the embodiments described above, the positions of the image-capturing unit 25 in a stopping state and in an idle state have not been described specifically, and the image acquisition device 20 may keep the image-capturing unit 25 waiting in a position away from a culture vessel during the stopping state and the idle state (that is, until just before image-capturing is started). The reason is that it is possible to prevent condensation from forming in the culture vessel by placing the image-capturing unit 25 which generates heat in the position away from the culture vessel. The position away from a culture vessel is not limited to a specific position, and is, for example, an end portion of a range in which the image-capturing unit 25 can be moved, such as an upper right corner.

What is claimed is:

1. A culture monitoring system comprising:
an image acquisition device that is arranged in an incubator, includes an image sensor that generates an image signal, and acquires, using the image sensor, an image of a biological sample cultured in the incubator; and
a controller that includes a processor and controls the image acquisition device, wherein
the processor
estimates a state of a temperature of the biological sample with respect to a set temperature of the incubator, and
restricts an operation of the image acquisition device at least according to the estimated state.

2. The culture monitoring system according to claim 1, wherein
at least according to the estimated state of the temperature of the biological sample, the processor causes the image acquisition device to transition from an operation state to a stopping state, the operation state being a state in which an image is able to be acquired, the stopping state being a state in which power consumption is lower than in the operation state.

3. The culture monitoring system according to claim 2, wherein
the processor transmits a stopping instruction indicating a stopping time period to the image acquisition device at least according to the estimated state of the temperature of the biological sample,
when the image acquisition device receives the stopping instruction, the image acquisition device transitions from the operation state to the stopping state, and
after the stopping time period indicated by the stopping instruction has elapsed, the image acquisition device transitions from the stopping state to the operation state.

4. The culture monitoring system according to claim 1, wherein
the processor estimates the state of the temperature of the biological sample on the basis of a control history of the controller controlling the image acquisition device.

5. The culture monitoring system according to claim 2, wherein
the processor estimates the state of the temperature of the biological sample on the basis of a control history of the controller controlling the image acquisition device.

6. The culture monitoring system according to claim 3, wherein
the processor estimates the state of the temperature of the biological sample on the basis of a control history of the controller controlling the image acquisition device.

7. The culture monitoring system according to claim 4, wherein
on the basis of the control history, the processor counts a number of acquired images of the biological sample that are acquired by the image acquisition device, and
the processor estimates the state of the temperature of the biological sample on the basis of the counted number of acquired images.

8. The culture monitoring system according to claim 7, wherein
the biological sample includes a plurality of biological samples accommodated in a plurality of regions obtained by partitioning a culture vessel, and
the processor restricts the operation of the image acquisition device at least according to the estimated state of the temperature of the biological sample and the number of image acquisition points set in the plurality of regions.

9. The culture monitoring system according to claim 4, wherein
the processor
determines, on the basis of the control history, whether an accumulated operation time obtained by accumulating operation times of the image acquisition device reached a first specified time period before a second specified time period has elapsed since a reference time, and
estimates the state of the temperature of the biological sample on the basis of a result of the determination of whether the first specified time period was reached.

10. The culture monitoring system according to claim 4, wherein
the processor
determines, on the basis of the control history, whether a weighted accumulated operation time obtained by accumulating operation times of the image acquisition device that have been weighted according to power consumption reached a first specified time period before a second specified time period has elapsed since a reference time, and
estimates the state of the temperature of the biological sample on the basis of a result of the determination of whether the first specified time period was reached.

11. The culture monitoring system according to claim 1, wherein
the processor estimates the state of the temperature of the biological sample on the basis of an image acquired by the image acquisition device.

12. The culture monitoring system according to claim 2, wherein
the processor estimates the state of the temperature of the biological sample on the basis of an image acquired by the image acquisition device.

13. The culture monitoring system according to claim 3, wherein
the processor estimates the state of the temperature of the biological sample on the basis of an image acquired by the image acquisition device.

14. The culture monitoring system according to claim 11, wherein
the processor estimates the state of the temperature of the biological sample on the basis of an image acquired by the image acquisition device, the image acquired by the image acquisition device being the image of the biological sample.

15. The culture monitoring system according to claim 11, wherein
the image acquisition device includes a member whose color is changed according to a temperature, and
the processor estimates the state of the temperature of the biological sample on the basis of an image of the member that is acquired by the image acquisition device.

16. The culture monitoring system according to claim 1, wherein
the image acquisition device includes a temperature sensor that measures a temperature, and
the processor estimates the state of the temperature of the biological sample on the basis of the temperature measured by the temperature sensor.

17. The culture monitoring system according to claim 2, wherein
the image acquisition device includes a temperature sensor that measures a temperature, and
the processor estimates the state of the temperature of the biological sample on the basis of the temperature measured by the temperature sensor.

18. The culture monitoring system according to claim 3, wherein
the image acquisition device includes a temperature sensor that measures a temperature, and
the processor estimates the state of the temperature of the biological sample on the basis of the temperature measured by the temperature sensor.

19. A method for controlling an image acquisition device arranged in an incubator, the method comprising:
estimating a state of a temperature of a biological sample with respect to a set temperature of the incubator; and
restricting an operation of the image acquisition device at least according to the state, the image acquisition device acquiring an image of the biological sample cultured in the incubator.

20. A non-transitory computer-readable medium having stored therein a program that causes a controller to execute a process, the controller controlling an image acquisition device that is arranged in an incubator and acquires an image of a biological sample cultured in the incubator, the process comprising:
estimating a state of a temperature of the biological sample with respect to a set temperature of the incubator; and
restricting an operation of the image acquisition device at least according to the state.
